(19) United States
(12) Patent Application Publication
(10) Pub. No.: US 2010/0049253 A1
(43) Pub. Date: Feb. 25, 2010

(54) BOTTOM LOADING CONNECTOR FOR ATTACHING A SPINAL ROD TO A VERTEBRAL MEMBER

(75) Inventor: Keith MILLER, Germantown, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/195,177

(22) Filed: Aug. 20, 2008

(51) Int. Cl. A61B 17/70

(52) U.S. Cl. 606/264

(57) ABSTRACT

Connectors for attaching a spinal rod to an anchor. The connectors may include a receiver with a rod-receiving opening on a bottom side that leads into a receiver passage sized to receive the spinal rod. A elongate member may extend through an opening on the top side of the receiver, and may include a cut-out to receive the spinal rod. A grommet may be operatively connected to the receiver and include a grommet passage to receive the anchor. A disk may be positioned between the receiver and the grommet and may include an angled surface that is positioned on a side of the receiver passage. The elongate member may be movable along the first axis between extended and retracted positions with the cut-out positioned closer to the second axis in the retracted position than in the extended position. Moving the elongate member from the extended position to the retracted position with the spinal rod received in the cut-out may cause the disk to move along the second axis towards the grommet passage to fix the first axis in spaced relation relative to the grommet passage.

### BACKGROUND

The present application relates to connectors for securing a spinal rod to a vertebral member, and more particularly to connectors with bottom loading features for receiving a spinal rod.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve.

Spinal rods may be implanted to support and position vertebral members in one or more of these regions. The spinal rods extend along a section of the spine and are attached to the vertebral members with one or more anchors. The anchors are typically screwed into the posterior portions of a vertebral member and pass through the pedicles and a substantial portion of the vertebral bodies and therefore provide a fixed and durable connection. The spinal rods are then attached to the anchors creating a rigid stabilization structure. In most situations, one such structure is provided on each lateral side of the spine.

Connectors provide a structure for attaching the spinal rods to the anchors. The connectors including a first receiver to receive the spinal rod and a second receiver to receive the anchor. The connectors should be adjustable to accommodate the anchors and/or spinal rods at various angular positions. The connectors may also be configured to facilitate the attachment of the spinal rods and anchors during a surgical procedure.

### SUMMARY

The present application is directed to connectors to attach a spinal rod to an anchor. The connectors may include a receiver with a rod-receiving opening on a bottom side that leads into a receiver passage sized to receive the spinal rod. A elongate member may extend through an opening on the top side of the receiver, and may include a cut-out to receive the spinal rod. A grommet may be operatively connected to the receiver and include a grommet passage to receive the anchor. A disk may be positioned between the receiver and the grommet and may include an angled surface that is positioned on a side of the receiver passage. The elongate member may be movable along the first axis between extended and retracted positions with the cut-out positioned closer to the second axis in the retracted position than in the extended position. Moving the elongate member from the extended position to the retracted position with the spinal rod received in the cut-out may cause the disk to move along the second axis towards the grommet passage to fix the first axis in spaced relation relative to the grommet passage.

Other aspects of various embodiments of the connectors are also disclosed in the following description. The various aspects may be used alone or in any combination, as is desired.

### BRIEF DESCRIPTION OF THE DRAWINGS

FIG. 1 is a perspective view of a connector that attaches a spinal rod to an anchor according to one embodiment.

FIG. 2 is an exploded perspective view of a connector according to one embodiment.

FIG. 3 is a side view of a connector during a first stage of attachment to an anchor and a spinal rod according to one embodiment.

FIG. 4 is a side view of a connector during a second stage of attachment to an anchor and a spinal rod according to one embodiment.

FIG. 5 is a side view of a first disk according to one embodiment.

FIG. 6 is a side view of a first disk according to one embodiment.

FIG. 7 is a side view of a first disk according to one embodiment.

FIG. 8 is a side view of a first disk according to one embodiment.

### DETAILED DESCRIPTION

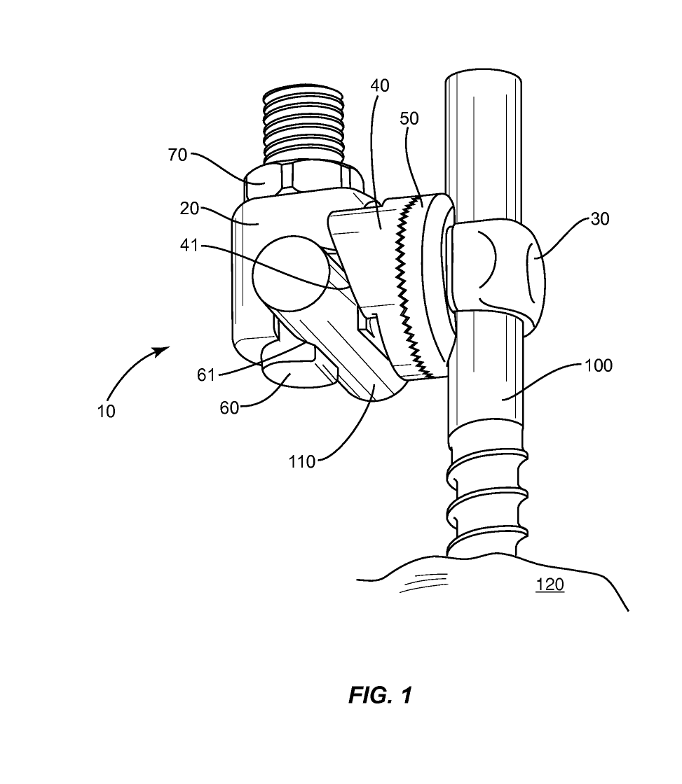

The present application is directed to connectors that connect a spinal rod to an anchor that is attached to a vertebral member. FIG. 1 illustrates one embodiment of a connector 10 that includes first and second disks 40, 50 positioned between and operatively connected to a receiver 20 and a grommet 30. The receiver 20 includes an elongate member 60 configured to receive a spinal rod 110, and the grommet 30 configured to receive an anchor 100. The disks 40, 50 are rotatable to adjust the angular position of the receiver 20 relative to the grommet 30. At the desired angular position, a nut 70 threaded on the elongate member 60 lifts the spinal rod 110 in the elongate member 60 into contact with an angled surface 41 of the first disk 40. This movement causes the disks 40, 50 to lock together and maintain the angular positions of the spinal rod 110 and anchor 100.

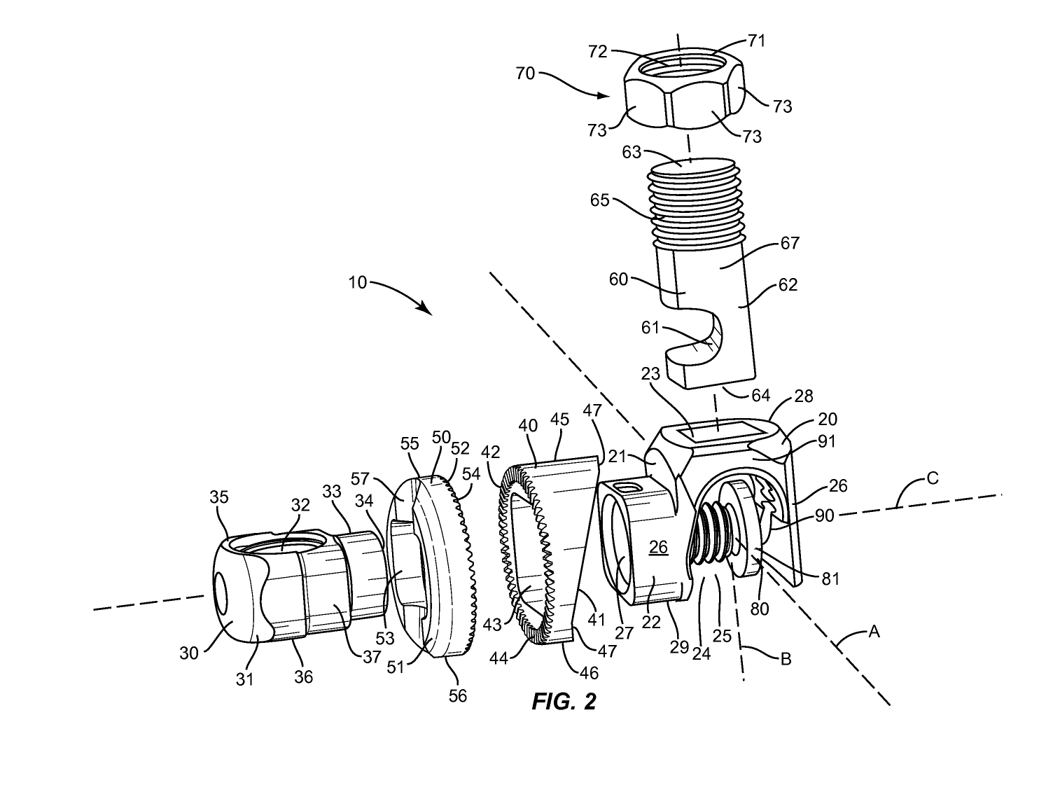

FIG. 2 includes an exploded view of a connector 10. The connector 10 the receiver 20, grommet 30, first disk 40, second disk 50, elongate member 60, and nut 70. These elements of the connector 10 act in concert to attach the spinal rod 110 to the anchor 100 in at a desired angular position. The connector 10 also provides for attaching the spinal rod 110 in a bottom-loading fashion due to the configurations of the receiver 20 and elongate member 60. This bottom-loading feature facilitates attachment of the spinal rod 110 to the anchor 100 during a surgical procedure. The connector 10 is configured for the spinal rod 110 to be offset from the anchor 100.

The receiver 20 includes a body 21 with a base 91, top side 28, bottom side 29, and lateral sides 26 therebetween. A passage 24 extends through opposing lateral sides 26 and forms a passage axis A. An opening 25 in the bottom side 29 leads into the passage 24 to allow for bottom-loading of the spinal rod 110. Bottom-loading is interpreted as the ability to insert the spinal rod 110 into the passage 24 through a lower section of the body 21. FIG. 2 includes the ability to insert the spinal rod 110 through the bottom side 29 of the body 21. A lateral wall 26 forms a section of the passage 24. Body 21 also includes an opening 23 through the base 91 that leads into the passage 24 through the top side 28 and is sized to receive the elongate member 60. Opening 23 forms an axis B that may be perpendicular to the passage axis A.

Body 21 further includes a neck 22 that extends outward from a lateral side 26 between the top and bottom sides 28, 29. The neck 22 includes a smaller height measured between the top and bottom sides 28, 29 than a main section of the body 21. The neck 22 may further include a substantially polygonal cross-sectional shape to engage with the first disk 40. FIG. 2 includes the neck 22 with a rectangular cross-sectional shape with rounded corners. An opening 27 extends through the neck 22 and into the passage 24. Opening 27 forms an axis C that may be perpendicular to one or both of axes A and B.

The grommet 30 includes a top side 35, bottom side 36, and lateral sides 37. A passage 32 extends through the grommet 30 between the top and bottom sides 35, 36 and is sized to receive the anchor 100. A neck 33 may extend outward from a lateral side 37 between the top and bottom sides 35, 36. In the embodiment of FIG. 2, neck 33 includes a circular cross-sectional shape that is sized to fit within the opening 27 in the receiver neck 22. The lateral sides 37 may include a polygonal cross-sectional shape.

The first disk 40 is positioned adjacent to the receiver 20. First disk 40 includes a first side 41 that faces towards the receiver 20, a second side 42 that faces away from the receiver 20, a top side 45, and a bottom side 46. The first side 41 is angled relative to the second side 42 such that a thickness of the first disk 40 increases from the bottom side 46 towards the top side 45. The first side 41 may be angled across the entire surface, or may include non-angled sections 47, such as those adjacent to the top and bottom sides 45, 46 as illustrated in FIG. 2. The angle of the first side 41 may be continuous, or may include sections of differing degrees of angulation. A passage 43 extends through the first disk 40 in the direction of the first and second ends 41, 42. The passage 43 is sized to receive the receiver neck 22. Passage 43 may include a polygonal shape to prevent relative rotation between the first disk 40 and the receiver 20. The second side 42 includes surface features 44 that engage with the second disk 50.

The second disk 50 is positioned between the grommet 30 and the first disk 40. The second disk 50 includes a first side 51 that faces towards the grommet 30, and second side 52 that faces away from the grommet 30, a top side 55, and a bottom side 56. A passage 53 extends through the grommet 30 between the first and second sides 51, 52. Passage 53 may include a polygonal shape that matches the shape of the lateral sides 37 of the grommet 30. The first side 51 may include a scalloped recess 57 that extends between the top and bottom sides 55, 56 to receive a section of the anchor 100 as will be explained in detail below. The second side 52 may include surface features 54 that engage with the first disk 40.

The surface features 44, 54 may include various sizes and configurations including but not limited to splines and teeth. The surfaces features 44, 54 may be complementary such that they mate together to lock the orientation of the connector 10. The surface features 44, 54 may extend across the entirety or one or more limited sections of the respective second sides 42, 52.

The elongate member 60 extends through the receiver 20 to contact the spinal rod 110. Elongate member 60 includes an elongated body 62 with a first end 63 and a second end 64. A cut-out section 61 is positioned towards the second end 64 and is shaped and sized to contact against the spinal rod 110. Threads 65 are positioned at the first end 63 to engage with the nut 70. The elongate member 60 is sized to extend through the opening 23 in the receiver 20 and position the cut-out 61 in or beyond the passage 24 to engage with the spinal rod 110. The cut-out 61 may include a continuous curve to extend around a majority of the surface of a spinal rod 110 with a circular cross-sectional shape.

The nut 70 includes a central opening 71 that includes threads 72 configured to engage with the threads 65 on the elongate member 60. A width of the nut 70 measured between lateral sides 73 may be greater than a width of the opening 23 for the nut 70 to be positioned on the top side 28 of the receiver 20 as illustrated in FIG. 1. The lateral sides 73 may further form a polygonal shape to engage with a driving tool.

In use, the various elements may be attached together prior to attachment with either of the anchor 100 or spinal rod 110. The neck 22 of the receiver 20 is sized to extend through the passage 43 of the first disk 40. The first disk 40 is moved along the neck 22 to an extent that the first end 41 may be positioned along each lateral side of the passage 24. The polygonal shapes of the neck 22 and passage 43 prevent relative rotation of these elements. The neck 33 of the grommet 30 is also sized to fit within the passage 53 of the second disk 50. The grommet 30 is inserted an extent such that the passage 53 contacts against the lateral sides 37. The polygonal shapes of the passage 53 and the lateral sides 37 prevent relative rotation between the grommet 30 and the second disk 50.

The neck 33 is further sized to extend outward beyond the second disk 50 an amount to extend into the opening 27 in the receiver 20. In this orientation, the first and second disks 40, 50 and the grommet 30 are aligned along the axis C that extends through the opening 27 of the receiver 20. As illustrated in FIG. 2, a fastener 80 may be positioned in the passage 24 and inserted through the neck 23, first and second disks 40, 50 and engage with threads on the opening 34 of the grommet 30. A head 81 of the fastener 80 is positioned in the neck 22 and away from the passage 24. An opening 90 may be positioned in a lateral side 26 of the receiver opposite from the neck 22. The opening 90 provides access to the head 81 by a drive tool to rotate and engage the fastener 80 in to the grommet 30.

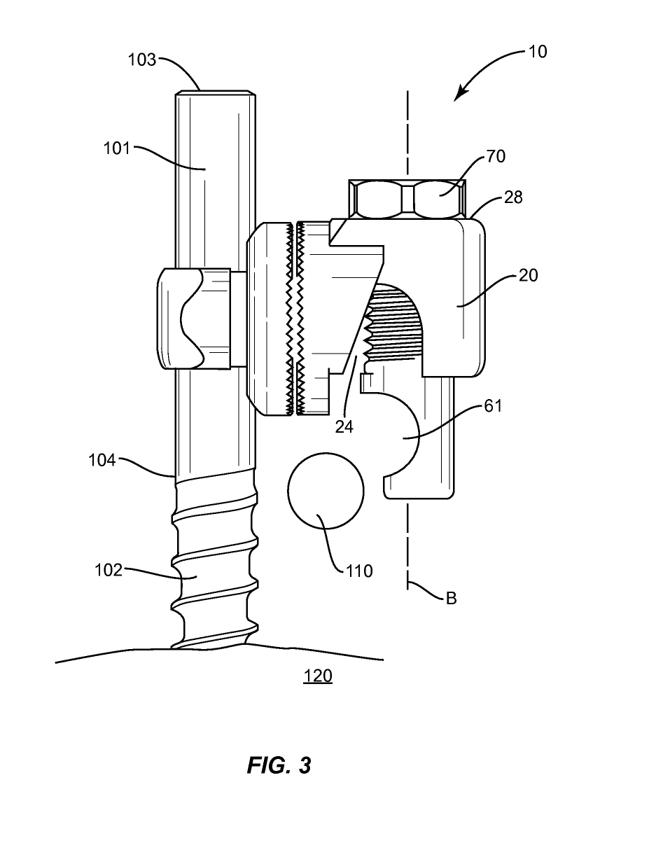

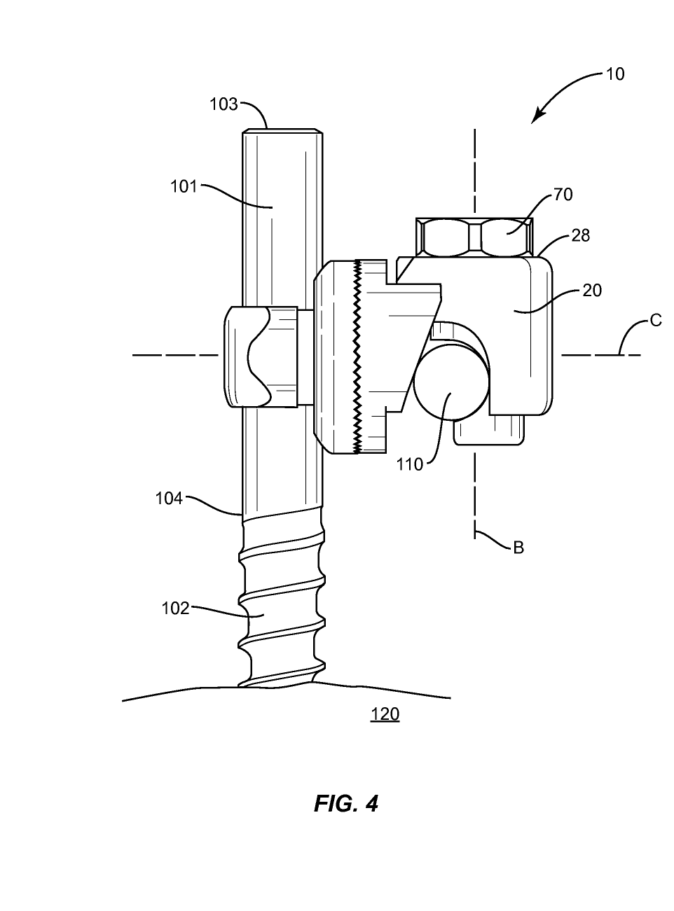

Once the elements of the connector 10 are attached together, the connector 10 may be attached to the anchor 100 and spinal rod 110. FIGS. 3 and 4 illustrate one method of attachment. The anchor 100 includes a threaded section 102 that mounts within a vertebral member 120, and a non-threaded section 101 that extends outward from the vertebral member 120. The non-threaded section includes a first end 103 at the top of the anchor 100, and a second end 104 at the start of the threaded section 102. The passage 32 in the grommet 30 is sized to receive the non-threaded section 101. The passage 32 is larger than the non-threaded section 101 for the grommet 30 to move along the height of the non-threaded section 101 between the first and second ends 103, 104. Further, the grommet 30 can be rotated about the longitudinal axis of the anchor 100 to be positioned at the desired angular position.

The receiver 20, grommet 30, and disks 40, 50 loosely attached together to allow for relative rotation between the disks 40, 50. This rotation provides for the cut-out 61 to be positioned at the necessary angular position to receive the spinal rod 110.

As illustrated in FIG. 3, the elongate member 60 is positioned in the receiver 20 with the cut-out 61 beyond the passage 24. The cut-out 61 faces towards the first disk 40 and is sized to extend around a portion of the spinal rod 110. The cut-out 61 may be shaped to match the cross-sectional shape of the spinal rod 110 to facilitate engagement. FIG. 3 includes the cut-out 61 sized to extend about half-way around the spinal rod 110. The elongate member 60 is sized to extend through the top side 28 of the receiver 20 to be engaged by the nut 70.

The spinal rod 110 is positioned in the cut-out 61 and the nut 70 is rotated about the elongate member 60. This rotation causes the nut 70 to move along the threads 65 and pull the elongate member 60 upward along axis B. Movement of the elongate member 60 moves the cut-out section 61 and engaged spinal rod 110 into the passage 24. As the elongate member 60 moves upward along axis B, the spinal rod 110 is moved into contact with the angled first end 41 of the first disk 40. This contact causes a force to be applied to the first disk 40 to move the first disk 40 along axis C and into engagement with the second disk 50. The engagement causes the surfaces 44 on the disks 40, 50 to contact together and lock the disks 40, 50 at the desired angular position.

The elongate member 60 and the receiver 20 may be keyed together to prevent rotation of the elongate member 60 as it axially moves through the receiver 20. FIG. 2 includes the elongate member 60 with a polygonal section 67 along the length between the threads 65 and the cut-out 61. The opening 23 may also include a polygonal shape. As the elongate member 60 moves axially through the opening 23, the polygonal section 67 mates with the polygonal opening 23 to prevent relative rotation between the elongate member 60 and the receiver 20. In another embodiment, the elongate member 60 and receiver 20 may be keyed through one or more notches that extend axially along the surface of the elongate member 60 that are sized to receive a tab that extends outward from the receiver 20. The notches may extend the entirety or a limited length of the elongate member 60. Alternatively, the elongate member 60 may include one or more tabs and the receiver 20 includes corresponding notches.

The upward movement of the elongate member 60 positions the spinal rod 110 between the cut-out 61 and the first end 41 as illustrated in FIG. 4. This captures the spinal rod 110 and prevents escape. The position of the spinal rod 110 along the first end 41 may vary depending upon the amount of angle of the first end 41. FIG. 4 includes the spinal rod 110 positioned with a center of the spinal rod 110 positioned below the axis C when the spinal rod 110 is in a locked position.

The force applied by the upwardly-moving spinal rod 110 may also cause the second disk 50 to move along the grommet 30. This movement may cause the scalloped recess 57 to engage with the non-threaded section 101 of the anchor. Alternatively, the scalloped recess 57 may be engaged with the non-threaded section 101 during attachment of the fastener 80.

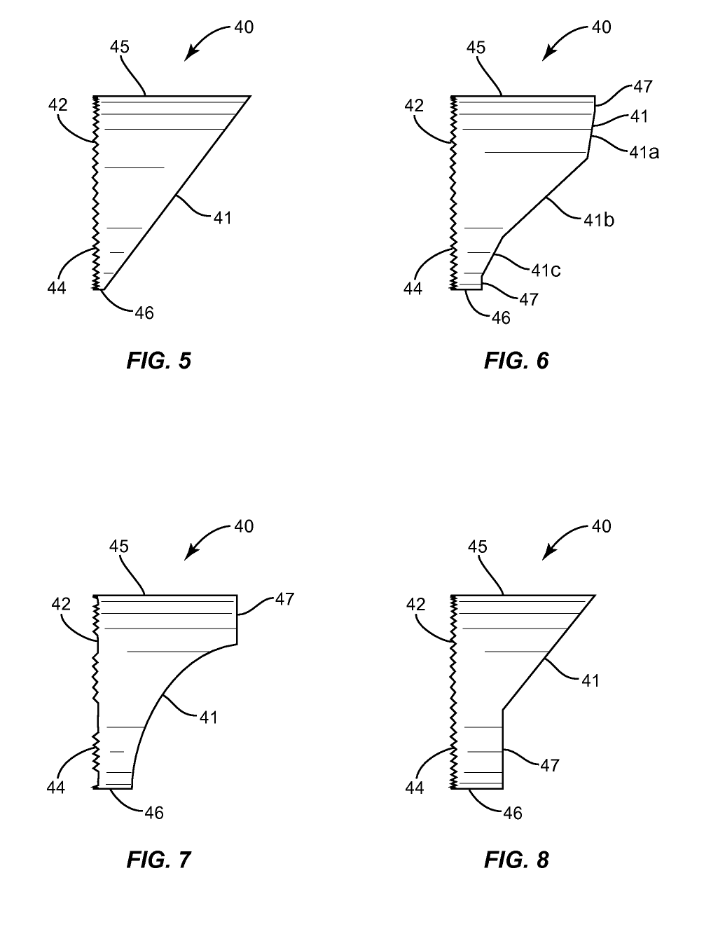

The first disk 40 may include various shapes. FIG. 5 includes the first end 41 including a constant angle between the top and bottom sides 45, 46. FIG. 6 includes the first side including sections of varying degrees with each of sections 41a, 41b, and 41c being angled a different amount. Non-angled lips 47 are positioned at each of the top and bottom sides 45, 46. FIG. 7 includes a curved surface 41. FIG. 8 includes an embodiment with the angled surface 41 positioned away from the bottom side 46.

The connector 10 may also include a single disk 40 positioned between the receiver 20 and grommet 30. The single disk 40 includes an angled surface that contacts the spinal rod 110 during movement of the elongate member 60. The opposite surface may include a scalloped recess similar to recess 57 to contact against the anchor 100. In use, the spinal rod 110 causes the single disk to move along the axis C and into contact with the anchor 100. The single disk may contact against the anchor 100 with enough force to lock the relative positions of the receiver 20 and grommet 30.

Embodiments of connectors 10 are disclosed in U.S. Pat. No. 7,261,715 and U.S. Patent Publication No. 2007/01622008 each of which is herein incorporated by reference in their entirety.

Various manners of attaching the elements of the connector 1 0 together along axis C are also contemplated in the present invention. FIG. 2 includes a fastener 80. Other manners include the grommet 30 including a flanged end, washers that attach the neck 33 to the receiver 20, and other mechanical fasteners. U.S. Pat. No. 7,261,715 and U.S. Patent Publication No. 2007/01622008 disclose other methods that are incorporated herein by reference.

Rod 100 may be made from a suitably strong rigid material known in the art, such as titanium, or from a semi-rigid material such as PEEK, polyurethane, polypropylene, or polyethylene. Rod 100 may include a variety of cross-sectional shapes including but not limited to circular, rectangular, square, and oval. Depending upon the context of use, the rod 100 may be linear or non-linear. The passage 50 is designed and the convex surface 21 tapered to accommodate the various shapes of the rod 100.

In the embodiments described above, the elements of the connectors 10 are attached together prior to attachment to either the anchor 100 or the spinal rod 110. In other methods, one or more of the elements may be attached to the anchor 100 or spinal rod 110 prior to attachment of the connector elements. In one embodiment, the grommet 30 is attached to the anchor 100 prior to being attached to the second disk 50.

One type of anchor 100 is a screw as illustrated in FIGS. 1, 3, and 4. Anchors 100 may also include a hook with a curved lower section that attaches to a vertebral member 120. Anchors 100 may also include various other configurations for attachment to a vertebral member 120.

Spatially relative terms such as “under”, “below”, “lower”, “over”, “upper”, “upward”, “downward” and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements,

The present invention may be carried out in other specific ways than those herein set forth without departing from the scope and essential characteristics of the invention. Further, the various aspects of the disclosed device and method may be used alone or in any combination, as is desired. The disclosed embodiments are, therefore, to be considered in all respects as illustrative and not restrictive, and all changes coming within the meaning and equivalency range of the appended claims are intended to be embraced therein.

What is claimed is:

1. A connector to attach a spinal rod to an anchor comprising:
a receiver with a base and opposing spaced-apart arms, the receiver including a receiver passage formed between the base and the arms and further including an open side opposite from the base to receive the spinal rod, the base further including an opening that extends into the receiver passage and is positioned opposite from the open side, the receiver including a first axis that extends through the opening and the open side;
a grommet operatively connected to the receiver, the grommet including a grommet passage sized to receive the anchor;
a disk positioned between the receiver and the grommet, the disk including a distal side facing towards the grommet passage and a proximal side facing away from the grommet passage, the proximal side including a face orientated at an acute angle relative to the first axis, the disk movable along a second axis towards the grommet passage;
an elongate member extending through the opening and into the receiver passage, the elongate member including a cut-out sized to receive the spinal rod;
the elongate member being movable along the first axis between extended and retracted positions wherein the cut-out is positioned closer to the second axis in the retracted position than in the extended position;
wherein moving the elongate member from the extended position to the retracted position with the spinal rod received in the cut-out causes the disk to move along the second axis towards the grommet passage to fix the first axis in an offset configuration relative to the grommet passage.

2. The connector of claim 1, wherein the disk includes a top side and a bottom side, the top side positioned in closer proximity to the base of the receiver than the bottom side when the disk is operatively connected to the receiver, the disk including a thickness measured between the distal side and the face that is larger towards the top side than the bottom side.

3. The connector of claim 1, wherein the receiver further includes a neck that extends outward from a lateral side between the base and the open side and the disk further includes a disk passage that extends through the distal and proximal sides, the neck positioned within the disk passage to operatively connect the disk and the receiver with the face of the disk oriented at the acute angle being aligned with the receiver passage to contact with the spinal rod when the elongate member moves from the extended position to the retracted position.

4. The connector of claim 3, further including the neck and the disk passage including polygonal cross-sectional shapes to prevent the disk from rotating relative to the receiver when the disk moves along the second axis.

5. The connector of claim 1, wherein the face of the disk orientated at the acute angle includes a first section oriented at a first acute angle and a second section oriented at a different second acute angle.

6. The connector of claim 1, wherein the cut-out is positioned in proximity to an end of the elongate member and includes a continuously curved surface.

7. The connector of claim 1, further including a nut positioned on a top side of the receiver and being connected to a threaded section of the elongate member that is positioned away from the cut-out.

8. The connector of claim 1, further including a second disk positioned between the disk and the grommet, each of the disk and the second disk including contact surfaces that engage together when the disk moves along the second axis towards the grommet passage to fix the first axis in the offset configuration relative to the grommet passage.

9. The connector of claim 1, wherein the first axis is perpendicular to the second axis.

10. A connector to attach a spinal rod to an anchor comprising:
a receiver with a base and a pair of arms with distal ends that are spaced apart to form a rod-receiving opening that leads into a receiver passage formed between the base and the arms, the base further including a second opening in the base that extends into the receiver passage, the receiver including a first axis that extends through the second opening and the open side;
a grommet operatively connected to the receiver, the grommet including a grommet passage sized to receive the anchor;
a first disk operatively connected to a lateral side of the receiver, the first disk including a top side and a bottom side with the top side positioned in closer proximity to the base of the receiver and the bottom side positioned in closer proximity to the rod-receiving opening, the first disk also includes a distal side facing towards the grommet passage and a proximal side facing away from the grommet passage, the proximal side including a face orientated at an acute angle relative to the first axis, the first disk including a thickness measured between the face and the distal side that is larger towards the top side than the bottom side, the first disk movable along a second axis towards the grommet passage;
a elongate member extending through the second opening and into the receiver passage, the elongate member including a cut-out to receive the spinal rod, the elongate member being movable along the first axis between extended and retracted positions wherein the cut-out is positioned closer to the second axis in the retracted position than in the extended position;
wherein moving the elongate member from the extended position to the retracted position with the spinal rod received in the cut-out causes the first disk to move along the second axis towards the grommet passage to fix the first axis in an offset configuration relative to the grommet passage.

11. The connector of claim 10, further including a second disk positioned between the first disk and the grommet, each of the first and second disks including splines on contact surfaces that mate together when the first disk moves along the second axis towards the grommet passage.

12. The connector of claim 11, wherein each of the first and second disks are rotatable relative to each other and non-rotatable relative to the receiver and the grommet.

13. The connector of claim 10, wherein the receiver further includes a neck that extends outward from the side of the receiver between the base and the rod-receiving opening and the first disk further includes a disk passage that extends through the distal and proximal sides, the neck positioned within the disk passage to operatively connect the first disk and the receiver with the face of the disk that is oriented at the acute angle being aligned with the receiver passage to contact with the spinal rod when the elongate member moves from the extended position to the retracted position.

14. The connector of claim 10, wherein the face of the first disk orientated at the acute angle includes a first section oriented at a first acute angle and a different second section oriented at a second acute angle.

15. The connector of claim 10, wherein the elongate member includes an elongated shape with a first end and a second end, the first end positioned to extend outward beyond a top side of the base and include a threaded section and the cut-out is positioned in proximity to the second end and includes a continuously curved surface.

16. The connector of claim 15, further including a nut positioned on the top side of the base and including a threaded opening that connects to the threaded section of the elongate member.

17. The connector of claim 10, wherein the first axis is perpendicular to the second axis.

18. A connector to attach a spinal rod to an anchor comprising:
a receiver with a base at a top side and opposing arms that are spaced apart to form a rod-receiving opening on a bottom side that leads into a receiver passage, the base including a second opening that leads into the receiver passage;
a first axis that extends through the second opening, the receiver passage, and the rod-receiving opening of the receiver;
an elongate member that extends through the second opening in the base and along the first axis, the elongate member also includes a cut-out sized to receive the spinal rod;
a grommet operatively connected to the receiver, the grommet including a grommet passage sized to receive the anchor;
a disk operatively connected to the receiver and including a proximal side and an opposite distal side that faces towards the grommet passage, the proximal side including an angled surface oriented at an acute angle relative to the first axis with a thickness of the disk measured between the angled surface and the distal side increasing from a bottom side of the disk towards a top side of the disk, the disk also being movable along a second axis towards the grommet passage;
the elongate member being movable along the first axis between extended and retracted positions wherein the cut-out is positioned closer to the second axis in the retracted position than in the extended position;
wherein moving the elongate member from the extended position to the retracted position with the spinal rod received in the cut-out causes the disk to move along the second axis towards the grommet passage to fix the first axis in spaced relation relative to the grommet passage.

19. The connector of claim 18, wherein a space formed between a bottom edge of the cut-out and a bottom edge of the angled surface of the disk being greater when the elongate member is in the extended position than when the elongate member is in the retracted position.

20. A method of attaching a spinal rod to an anchor comprising:
extending an elongate member through an opening in a receiver and along a first axis;
positioning the spinal rod in a cut-out in the elongate member when the cut-out is positioned away from a receiver passage formed by a base and opposing arms of the receiver;
threading a nut onto a threaded section of the elongate member and moving the elongate member and the spinal rod along the first axis and into a rod-receiving opening in the receiver formed between distal ends of the opposing arms;
contacting the spinal rod against an angled surface of a disk that is operatively connected to the receiver and moving the disk away from the receiver passage and towards the anchor that is positioned within a grommet;
moving the elongate member and the spinal rod further along the first axis and continuing to move the disk father away from the receiver passage;
capturing the spinal rod in the receiver passage against the angled surface and locking the angular position of the receiver relative to the grommet to position the anchor at the desired angular position relative to the anchor.

21. The method of claim 20, further comprising contacting the disk against a second disk that is movably positioned on the grommet.

22. The method of claim 20, further comprising contacting the nut against a top surface of the receiver as the elongate member moves along the first axis.

23. The method of claim 20, wherein moving the disk away from the receiver passage includes moving the disk along a second axis that is perpendicular to the first axis.
